(19) United States
(12) Patent Application Publication
(10) Pub. No.: US 2003/0212423 A1
(43) Pub. Date: Nov. 13, 2003

(54) Analyte test element with molded lancing blade

(76) Inventors: Jerry T. Pugh, Mountain View, CA (US); Keichi Aoyagi, Los Altos, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/143,129

(22) Filed: May 9, 2002

(51) Int. Cl. A61B 17/32

(52) U.S. Cl. 606/181

(57) ABSTRACT

Devices, systems and methods are provided for cutting the skin, accessing and collecting physiological sample therein, and measuring a characteristic (e.g., an analyte concentration, of the sampled physiological sample). The subject devices are in the form of a tester or test element which includes a biosensor and preferably only one blade member that is removed in location from the biosensor. The placement of the blade or micro-blade element may vary, but is most preferably located to provide clearance from a user's finger during biological sample collection in connection with the biosensor. The testers are preferably configured to be stacked in a magazine for successive firing by a meter configured to automatically actuate sample acquisition and reading. Systems and methods in connection with the subject testers are also provided.

### FIELD OF THE INVENTION

This invention relates to collection of physiological samples and the determination of analyte concentrations therein, especially where the biological samples are whole blood or interstitial fluid.

### BACKGROUND OF THE INVENTION

Analyte concentration determination in physiological samples is of ever increasing importance to today's society. Such assays find use in a variety of application settings, including clinical laboratory testing, home testing, etc., where the results of such testing play a prominent role in the diagnosis and management of a variety of disease conditions. Analytes of interest include glucose for diabetes management, cholesterol for monitoring cardiovascular conditions, and the like. In response to this growing importance of analyte concentration determination, a variety of analyte concentration determination protocols and devices for both clinical and home testing have been developed.

In determining the concentration of an analyte in a physiological sample, a physiological sample must first be obtained. Obtaining the sample often involves cumbersome and complicated devices that may not be easy to use or may be costly to manufacture. Furthermore, the procedure for obtaining the sample is often relatively painful.

The level of pain is usually associated with the size of the needle used to obtain the physiological sample and the depth to which the needle is inserted. Depending on the analyte and the type of test employed, a relatively large, single needle or the like is often used to extract the requisite amount of sample. Alternately, certain systems for obtaining biological sample teach the use of a plurality of micro-needle or micro-piercing members. Examples of such are disclosed in PCT publications: WO 00/64580; WO 00/74763; WO 00/74764 and WO 00/35530. The latter publication even shows a test strip employing a micro needle assembly backed by a layer of material including reagent for determining the concentration of analyte present in the sample take. It is also contemplated that the piercing member may take various shapes. Certain embodiments disclosed in '530 publication as well as in the other references may be made of plastic with the piercing elements formed integrally with a substrate.

Other references disclose the use of blades in order to reduce the pain associated with obtaining a sample. These include U.S. Pat. Nos. 5,314,441; 5,395,387 and 5,476,474. The devices in the '441 and '474 patents employ a translational or swiping motion to produce a cut like a scalpel in order reduce pain as compared to a needle/lancet stick. The '387 patent provides for a puncture-style device, but deceases the pain associated with such action by angling the cutting edge of the blade in order to produce greater shear resulting in improved slicing action and less pressure at sample site.

Additional devices that employ blade-type structures—as opposed to needle-type structures—are presented in U.S. Pat. Nos. 5,212,879 and 5,529,581. Like the device in the '387 patent, these too employ puncture-type actuation.

In all, each of the systems in the U.S. Pat. Nos. 5,314,441; 5,395,387 and 5,476,474 and. 5,212,879 and 5,529,581 fail to meet certain needs addressed by the present invention. Each of the referenced systems merely represent lancing mechanisms. Accordingly, they offer no appreciable benefit in simplifying testing procedures.

What is more, the other systems noted above that offer combined test strip and lancing functionality fail to provided the additional advantages offered by the present invention. Namely, test strips according to the present invention are adapted for use in an automated or semi-automated system to quickly and easily lance a site and transfer biological fluid to a remotely situated test element.

While certain combination test strip and lancing systems do exist (see U.S. Pat. Nos. 6,099,484 and 5,820,570) these systems are quite complex and, consequently, can be difficult to operate or costly to produce. As such, there is continued interest in the development of new devices and methods for use in the determination of analyte concentrations in a physiological sample.

The assignee of the present invention holds title to various other systems that possess certain advantages involving integrated test strip, lancing combinations (see U.S. patent application Ser. No. 09/923,093 and Attorney Docket No. 35). The present invention provides yet another viable option with such alternate advantages.

As will be apparent from the following description, the approach of the present invention is particularly viable in view of its ease of manufacture low cost construction and reliability and speed in use. Furthermore, it may be configured to obtain either a blood sample or an interstitial fluid sample and analyze the same. Though not every such advantage need be presented in every variation of the present invention, those will skill in the art may yet recognize further advantages in connection with the same.

### SUMMARY OF THE INVENTION

Devices, systems and methods for accessing, collecting a physiological sample and measuring a characteristic of the sample are disclosed. The subject systems include a tester with in integrally formed skin-cutting element integral and a biosensor, preferably in the form of a reagent pad. The elements are spaced apart from one another to make discrete the actions of producing a laceration or micro-laceration and collection biological fluid therefrom.

In some variations of the tester, the biosensor comprises electrochemical cell. Alternately, it may be a photometric or colorimetric biosensor having a planar substrate defining a photometric matrix area, optionally covered by a photometric membrane, collectively configured for receiving a sample to be tested. The subject biosensors are useful in the determination of a wide variety of different analyte concentrations, where representative analytes include, but are not limited to, glucose, cholesterol, lactate, alcohol, and the like. In many embodiments, the subject test strips are used to determine the glucose concentration in a physiological sample, (e.g., interstitial fluid, blood, blood fractions, constituents thereof, and the like).

A frame of the tester or test element is preferably configured so that a number may be stacked in a complimentary or interlocking manner. Most preferably, they so-stacked and employed as a magazine loaded into a test strip meter. The meter should be adapted to fire successive tester members until the magazine is spent as well as to obtain a reading from the biosensor indicative of a characteristic of the sampled fluid, (e.g., the concentration of at least one analyte in the sample).

Irrespective of details of the meter's physical and electronic operation, the testers are configured with a blade member that can be moved into and out of contact with the user to cleanly slice the skin. The present invention includes the subject devices and methodology, kits that include the subject devices and/or systems for use in practicing the subject methods, and also results or data produced according to the teachings of the present invention.

### BRIEF DESCRIPTIONS OF THE DRAWINGS

Each of the figures diagrammatically illustrates aspects of the invention. To facilitate understanding, the same reference numerals have been used (where practical) to designate similar elements that are common to the figures.

FIG. 1A is a top perspective view of a test element or tester according to the present invention; FIG. 1B is a detail view of a blade portion of the tester shown in FIG. 1A; FIG. 1C is a bottom perspective view of the tester shown in FIG. 1.

FIG. 2 shows electrochemical sensor elements that may be substituted for the colorimetric elements shown in FIG. 1A.

FIG. 3A is a top perspective view of the tester shown in FIGS. 1A and 1B; FIG. 3B is a bottom perspective view of the tester shown in FIGS. 1A and 1B in combination with a meter sensor element.

FIGS. 4A-4D are side views of an alternate tester shown at various stages in the subject methodology.

FIGS. 5 and 6 are a side view still further tester variations.

FIG. 7A is a perspective view of a ring for assisting in obtaining sample; FIG. 7B is a cross sectional view of the ring of FIG. 7A take along line A-A, shown in use with the tester of FIG. 5.

### DETAILED DESCRIPTION OF THE INVENTION

In describing the invention in greater detail than provided in the Summary above, one variation of the inventive tester is described. After introduction of the basic tester of the invention, optional sensor systems are described. Next, use of the tester in connection with a meter is disclosed. Following this description, an alternate tester is described in connection with its method of use.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, it is noted that unless defined otherwise below, all technical and scientific terms used herein have the same meaning as commonly understood by one of ordinary skill in the art to which this invention belongs.

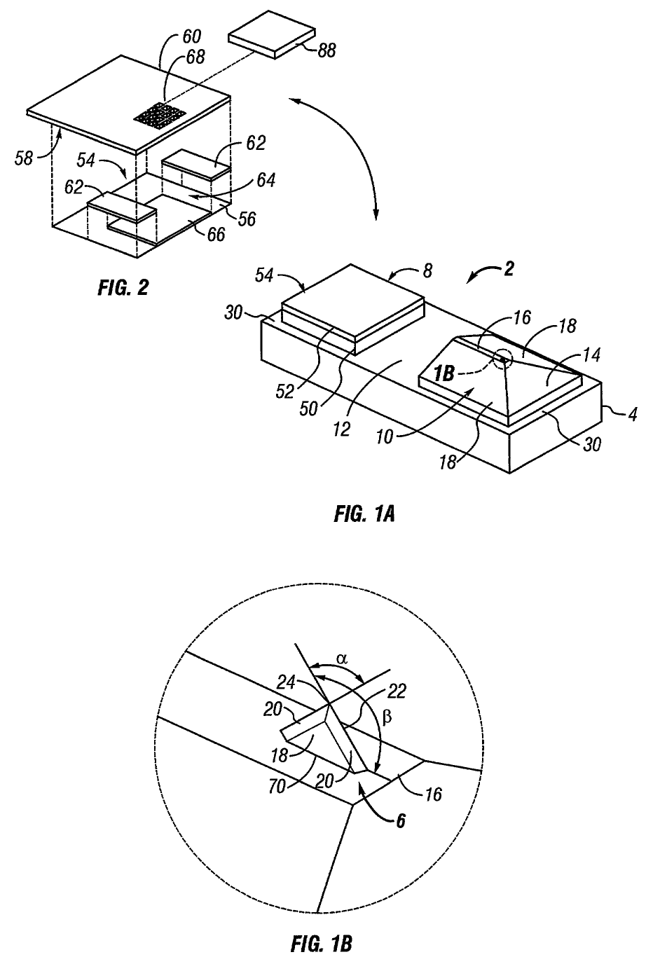

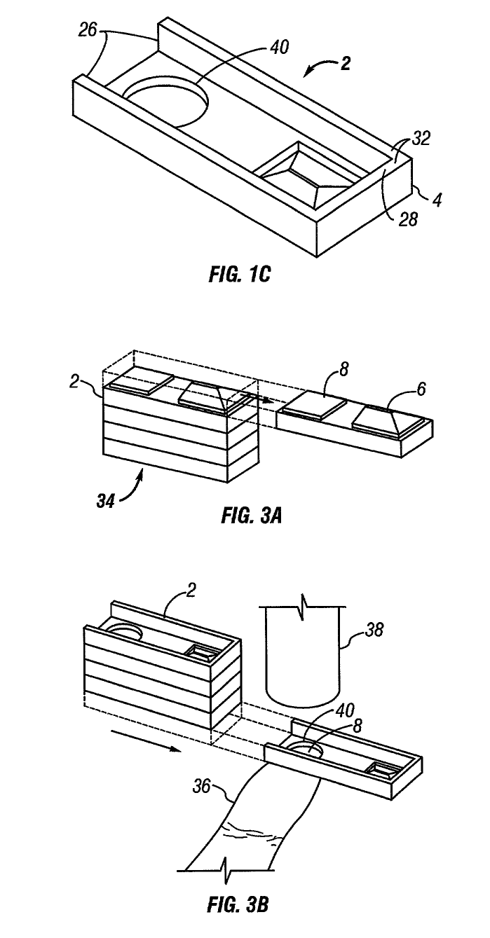

Turning now to FIGS. 1A-1C, a first test element or tester 2 is shown. It comprises a frame 4, a blade portion 6 and a sensor portion 8.

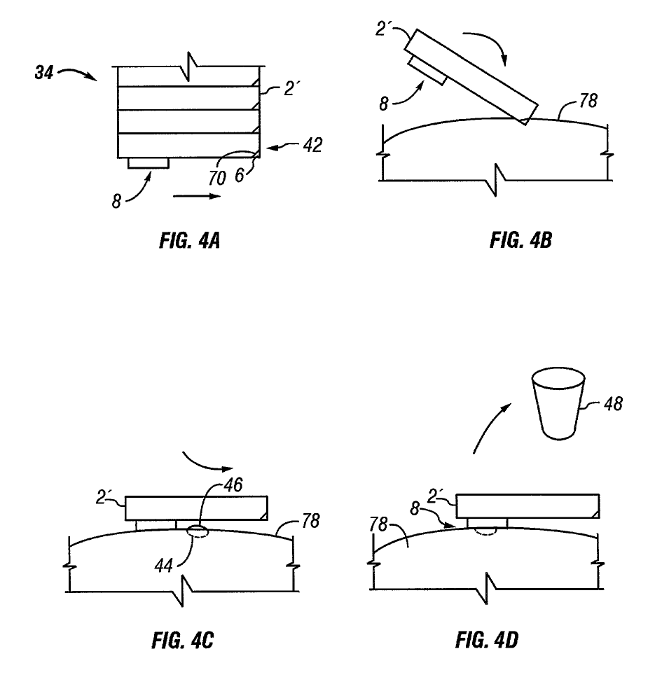

The blade is carried by frame 4. While in the variation of the invention shown in FIGS. 1A and 1B an intermediate wedge or raiser member 10 is provided between the blade and frame, in the variation of FIGS. 4A, 4B, 5 and 6 the blade is carried directly by the frame. The raiser may be integrally molded in frame 4 as indicated by its relieved back side.

Still, elevating the blade relative to a top surface or face 12 of the frame, particularly with a wedge having an angled front surface or ramp portion 14, may present certain advantages. Configured properly, it allows for greater pressure to be applied between the blade and the skin of a user while only using simple translational motion to actuate the tester. With a wedge or ramp member transitioning from a surface elevation of a tester at or about at the lever of the biosensor as shown in FIG. 1A, the tester is easily actuated in a horizontal action across a user's finger or another relatively raised area without significant concern regarding impediments to its motion.

Such action is illustrated in FIGS. 3A and 3B. As the wedge comes into contact with, for example, a users finger, the digit ramps up wedge front surface 14. The compression of tissue (when the finger is held in a relatively fixed location relative to face of the tester—perhaps against a base of a meter actuating the tester), results in pressure applied between the blade and finger. This can help produce a clean and virtually painless cut.

Actually, another feature of the wedge assists in avoiding impediment to actuation as well. A narrow land 16 is provided. As shown in detail view FIG. 1B, the blade may be located on the land. Since the land is relatively narrow (preferably between about 0.5 mm and 2.0 mm), the area over which frictional forces may act as an impediment to tester actuation is minimized.

Providing a narrow land feature with sides 18 that are angled or drop off with respect to the land, increases the ability to compress tissue riding over the structure. While the pressure contributes to frictional forces, the benefit of higher pressure at the blade/tissue interference tissue interface has already been noted.

Returning to the detail of the blade portion shown in FIG. 1B, a preferred configuration is illustrated. In the preferred configuration, a triangular member is used. However, other shapes may be employed. Whatever the blade shape selected, its geometry should be robust enough to avoid break-off. It is also the case that the shape should be chosen to facilitate production by injection molding techniques. The triangular shape, with its larger base and substantially vertical side portions (which actually preferably include some draft angle to facilitate recovery from a mould), is suited to each goal.

To provide a blade with sufficient thickness (t) to avoid break-off, instead of tapering the each side 18, it is preferred to use one or more champhered sections 20 to define an edge of the blade 22.

Champhered portions 20 may be paired so the blade edge(s) lies between the same. Alternately, a single champhered section may be used. In which case, blade edges 22 are provided as shown in FIG. 1B.

A point 24 is provided at the intersection of the champhered sections. Though such a point may be used in a piercing type action of the skin. A preferred approach involves a cutting or slicing action as described above and as further described below.

It may be the case that the blade only includes one sharp edge. In which case, it will be the leading edge 22. Further variation of various blade parameters is also possible. Variation is contemplated with respect to the overall angle it defines as viewed from the side. Generally, blades with profile having an angle a between 60 and 135° may be preferred. The angle P the leading edge of the blade makes with respect to land 16 (or other adjacent support structure) is preferably between 90° and 150°. In order to easily obtain a blood sample, blade 6 preferably has an overall height measured from a base 70 to point or apex 24 of between about 0.1 and 1.0 mm. Smaller blades may be suitable for obtaining interstitial fluid.

While variation of these blade parameters may vary, it is important that blade 6 can be produced by injection molding in order to keep minimize costs in volume production. Further, as noted above, strength concerns are important as well.

However the blade is configured, the tester frame preferably is configured for stacking a plurality of such items upon one another. To provide clearance for blade, wedge and/or sensor features, the frame preferably includes at least a pair of opposed side walls 26, though any approach to providing some sort of “standoff” from the adjacent sensors may be used. Further, a front wall 28 may be provided.

Features complimentary to the walls may be provided to help hold stacked testers in a stable location with respect to each other. Such features include the peripheries of wedge 10 and/or sensor 8. It is easily envisioned how the ledge or shoulder 30 provided around each such item can capture the base 32 of walls 26 or 28.

It is preferred, that the frame is left open at or on the sensor side. Such a frame configuration facilitates the action shown in FIGS. 3A and 3B. As seen from the top in FIG. 3A, a tester 2 arranged as part of a magazine 34 is able to slide off the stack from front to back, in line with the blade edge. With a finger set opposite the face of the tester as shown in FIG. 3B, a cut is made in passing the blade over the finger 36 shown. The sensor 8 is then placed opposite the cut to collect and analyze a sample that springs therefrom. Stated another way, the initial portion of the lateral motion causes the blade to engage the skin of the test subject to produce a wound; the remainder of the motion brings the test element into contact with the wound site.

Blood (or interstitial fluid) which exits the wound is then drawn into the test element, usually by capillary action produced by the small pores associated with the sensor element. The type of sample obtained depends largely on the size of the blade and depth of the wound it creates. A shallow wound that does not disrupt the capillaries (50 to 500 μm depending on the location of the lance site) could provide an interstitial fluid sample. A deeper cut will generally provide a blood sample. Extraction of the fluid sample may be facilitated by a pressure element which surrounds the wound site or palpation.

As shown in FIG. 7A the pressure element may take the form of a ring 72. In use, the ring is pressed against a tissue site to produce a bulge of stretched tissue 74. Such action produces a structure which is more easily cut to obtain sample; further, the pressure at the site urges fluid therefrom. Further details as to use of pressure ring 72 are provided below.

Where a calorimetric sensor is provided, a meter sensor 38 may be set opposite an aperture 40 in the tester to take readings. Alternately, the aperture may be omitted if a substantially transparent section of material backs the colorimetric sensor. Of course, the meter sensor type may vary depending on the nature of the biosensor 8.

In FIG. 3B, the tester is shown moving across the user's finger. In this manner, the blade is clear of tissue after a cut is made, including during sample collection and/or testing.

In the variation of the invention shown in FIGS. 4A-4D, 5 and 6 alternate provisions are made for tissue/blade clearance. These other variations are suited particularly suited for use in testing alternate sites. For example, the forearm or back of the hand may be employed as a test site. Taking action on a site other than a finger is highly advantageous from the perspective of minimizing pain since many such alternate locations do not have the same level of feeling as the fingers.

As in FIGS. 3A and 3B, a magazine of testers is provided. However, in each of testers 2′ at least one blade element 6 is provided at the junction the face and front wall of the frame 4. This positioning may be varied considerably so long as the blade clears a sample site according to the methodology shown by the sequential positions and arrows indicating direction of movement of the tester 2′ in each of FIGS. 4A-4D. For instance, one or more blades 6 may be provided that project outwardly from the front wall only, or be constructed as shown where adjacent relieved portions 42 expose blade 6.

After sliding out from a magazine as shown in FIG. 4A, a tester 2′ is shown being tilted or angled forward so blade 6 can produce a wound in a subject's finger 36, forearm 78 (or another site) from which sample may be obtained. As it progresses forward as shown in FIG. 4C, the tester levels-out. The wound 44 produced and pooling sample 46 leaving the test site can also be seen (though the sample may not initially flow in such manner in the time it takes to actuate the tester).

In FIG. 4D after such lateral movement is complete, sample is taken-up by sensor 8, and testing for analyte concentration proceeds. Preferred types of sensors and testing that may be accomplished are treated below. Once testing is complete and the tester is spent, it is typically disposed of in an appropriate garbage receptacle 48.

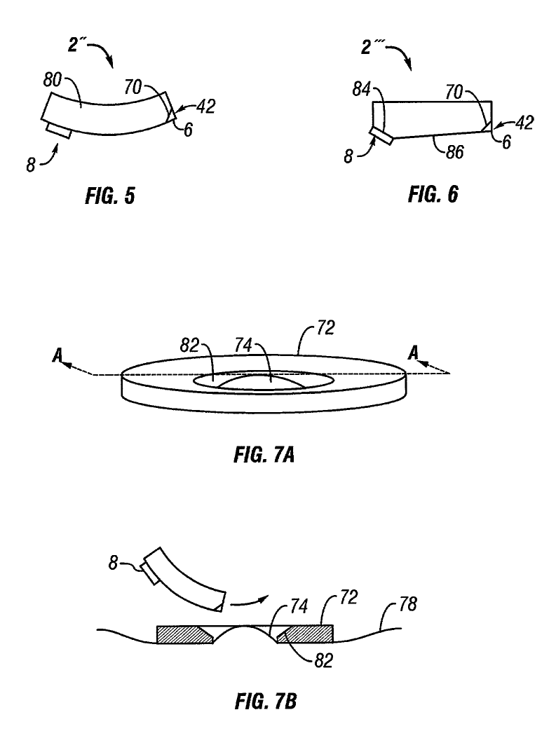

Alternate tester configurations are shown in FIGS. 5 and 6. The tester 2″ in FIG. 5 has a curved body 80. When stacked with other test elements (in a similar manner to those described above), the curved aspect facilitates removal of elements along a curve, rather than straight-line path. The action of the device in producing a wound and, ultimately, for collecting and testing a sample is shown in FIG. 7B. Here, the arcuate motion of the device is depicted. As shown, blade portion 6 will contact tissue site 74 to produce a wound. Then, tester 2″ continues around to place test element 8 in contact with the wound location from which sample springs. To facilitate this action, ring 72 includes relieved sections 82 for added clearance. In any case, mounting the sensor portion 8 substantially as shown provides clearance for the blade portion 6 when the former is in contact with a wound site.

The tester 2′″ in FIG. 6 may be used in a similar fashion. However, instead of mounting the sensor member along a curved body to provide blade/sensor clearance, a separate angled mounting surface 84 for the sensor is provided. The tester may include a contiguous surface 86 or the device may be configured otherwise. In any case, the angled mounting surface 84 provides for a convex or outwardly-facing sensor with respect to frame 4, like the arrangement in FIG. 5.

In each of the approaches of tester use described, a meter system (of which, the sensor portion is shown in FIG. 3B) holds the components in their respective locations and produces the necessary motions to accomplish a test. During the process of cutting, sampling and testing, the meter is preferably held stationary against the test site on the patient.

The tester movements described above preferably occur automatically by action of the meter and an included actuator as a result of some simple user action. For example, the motions could occur when user pushes a button on the meter or simple presses the meter against the test site. As for the workings of a meter able to produce the desired action, the design and production of certain actuators is well within the level of skill in the art. Yet, it may be the case that creation of certain other actuators may represent inventive activity.

Manufacture

In producing testers according to the present invention, the frame and blade are preferably injection molded as an integral piece of plastic. However, it may be the case that the wedge and blade are one molded element and the frame another. In which case, any suitable adhesive or welding approach (e.g., chemical or ultrasonic welding) may be utilized to join the elements. Material selection may vary depending on the approach taken. However, in any element which includes blade 6, suitable materials that may be used for molding the same. One such material is liquid crystal polymer (e.g., Vectra® material available from Tiucona, 90 Morris Avenue, Summit, N.J. 07901-3914).

Whatever the material selected and molding approach employed, certain aspects of the tester are required according to the present invention. One such aspect is the use of spaced apart or discrete locations for the blade and sensor member. By discrete, it is meant that the members are not interconnected by another structure such as a fluid pathway; they are disconnected or isolated members. The invention operates by movement of the tester relative to the sampling site. The tester is configured so that at one location it includes a means for creating a wound to access a biological sample, and at another location a sensor to receive sample from the wound. The sensor area which receives the sample incorporates an on-board reagent system for testing the sample for analyte concentration.

The sensor is preferably configured as an electrochemical cell or as a photometric or calorimetric biosensor. The former type is shown in connection with FIG. 2; the latter type, is shown in FIGS. 1A and 1C, though either type may be used.

Colorimetric/Photometric Sensor Variations

In testers including colorimetric or photometric (herein used interchangeably) biosensor, the same is provided by at least a matrix element 50 for receiving a sample, a reagent composition (not shown as a structural component, but set within matrix 50, and possibly also in an optional, topmost membrane 52. Where a membrane or other top layer is provided, it includes aperature or pores for sample access. Such pores may be extremely small, and thus, are not shown.

In some embodiments, top layer 52 may be a membrane containing a reagent composition impregnated therein while the matrix 50 may or may not contain reagent composition. Matrix 50 preferably provides a deposition area for the various members of the signal producing system, described infra, as well as for the light absorbing or chromogenic product produced by the signal producing system, i.e., the indicator, as well as provides a location for the detection of the light-absorbing product produced by the indicator of the signal producing system.

If a top layer is provided, it may be transparent so that the color intensity of the chromogenic product resulting from the reaction between the target analyte and the signal producing system can be measured. It should, however be permeable to sample fluid.

Alternately, top layer 52 may comprise a membrane that is of aqueous fluid flow and is sufficiently porous (i.e., provides sufficient void space) for chemical reactions of a signal producing system to take place. Ideally, the membrane pore structure would not support red blood cell flow to the surface of the membrane being interrogated (i.e., the color intensity of which is a subject of the measurement correlated to analyte concentration). Matrix 50 may or may not have pores and/or a porosity gradient, e.g. with larger pores near or at the sample application region and smaller pores at the detection region.

Materials from which matrix membrane 52 may be fabricated vary, include polymers, e.g. polysulfone, polyamides, cellulose or absorbent paper, and the like, where the material may or may not be functionalized to provide for covalent or non-covalent attachment of the various members of the signal producing system. In a tester made a thin membrane material, the tester may require less than ½ μl of sample to wet a sufficiently large area of the membrane to obtain a good optical measurement.

A number of different matrices have been developed for use in various analyte detection assays, which matrices may differ in terms of materials, dimensions and the like, where representative matrices include, but are not limited to, those described in U.S. Pat. Nos. 4,734,360; 4,900,666; 4,935,346; 5,059,394; 5,304,468; 5,306,623; 5,418,142; 5,426,032; 5,515,170; 5,526,120; 5,563,042; 5,620,863; 5,753,429; 5,573,452; 5,780,304; 5,789,255; 5,843,691; 5,846,486; 5,968,836 and 5,972,294; the disclosures of which are herein incorporated by reference.

The one or more members of the signal producing system produce a detectable product in response to the presence of analyte, which detectable product can be used to derive the amount of analyte present in the assayed sample. In the subject test strips, the one or more members of the signal producing system are associated (e.g., covalently or non-covalently attached to) at least a portion of (i.e., the detection region) the matrix, and in many embodiments to substantially all of the matrix.

The signal producing system is preferably an analyte oxidation signal producing system. By analyte oxidation signal producing system it is meant that in generating the detectable signal from which the analyte concentration in the sample is derived, the analyte is oxidized by a suitable enzyme to produce an oxidized form of the analyte and a corresponding or proportional amount of hydrogen peroxide. The hydrogen peroxide is then employed, in turn, to generate the detectable product from one or more indicator compounds, where the amount of detectable product generated by the signal measuring system, i.e. the signal, is then related to the amount of analyte in the initial sample. As such, the analyte oxidation signal producing systems present in the subject test strips are also correctly characterized as hydrogen peroxide based signal producing systems.

Hydrogen peroxide based signal producing systems include an enzyme that oxidizes the analyte and produces a corresponding amount of hydrogen peroxide, where by corresponding amount is meant that the amount of hydrogen peroxide that is produced is proportional to the amount of analyte present in the sample. The specific nature of this first enzyme necessarily depends on the nature of the analyte being assayed but is generally an oxidase or dehydrogenase. As such, the first enzyme may be: glucose oxidase (where the analyte is glucose), or glucose dehydrogenase either using NAD or PQQ as cofactor; cholesterol oxidase (where the analyte is cholesterol); alcohol oxidase (where the analyte is alcohol); lactate oxidase (where the analyte is lactate) and the like. Other oxidizing enzymes for use with these and other analytes of interest are known to those skilled in the art and may also be employed. In those preferred embodiments where the reagent test strip is designed for the detection of glucose concentration, the first enzyme is glucose oxidase. The glucose oxidase may be obtained from any convenient source (e.g. a naturally occurring source such as Aspergillus niger or Penicillum, or recombinantly produced).

The second enzyme of such a signal producing system is an enzyme that catalyzes the conversion of one or more indicator compounds into a detectable product in the presence of hydrogen peroxide, where the amount of detectable product that is produced by this reaction is proportional to the amount of hydrogen peroxide that is present. This second enzyme is generally a peroxidase, where suitable peroxidases include: horseradish peroxidase (HRP), soy peroxidase, recombinantly produced peroxidase and synthetic analogs having peroxidative activity and the like. See, e.g., Y. Ci, F. Wang; Analytica Chimica Acta, 233 (1990), 299-302.

The indicator compound or compounds, are preferably ones that are either formed or decomposed by the hydrogen peroxide in the presence of the peroxidase to produce an indicator dye that absorbs light in a predetermined wavelength range. Preferably the indicator dye absorbs strongly at a wavelength different from that at which the sample or the testing reagent absorbs strongly. The oxidized form of the indicator may be a colored, faintly-colored, or colorless final product that evidences a change in color of the testing side of the membrane. That is to say, the testing reagent can indicate the presence of glucose in a sample by a colored area being bleached or, alternatively, by a colorless area developing color.

Indicator compounds that are useful in the present invention include both one- and two-component chromogenic substrates. One-component systems include aromatic amines, aromatic alcohols, azines, and benzidines, such as tetramethyl benzidine-HCl. Suitable two-component systems include those in which one component is MBTH, an MBTH derivative (see e.g., those disclosed in U.S. patent application Ser. No. 08/302,575), or 4-aminoantipyrine and the other component is an aromatic amine, aromatic alcohol, conjugated amine, conjugated alcohol or aromatic or aliphatic aldehyde. Exemplary two-component systems are 3-methyl-2-benzothiazolinone hydrazone hydrochloride (MBTH) combined with 3-dimethylaminobenzoic acid (DMAB); MBTH combined with 3,5-dichloro-2-hydroxybenzene-sulfonic acid (DCHBS); and 3-methyl-2-benzothiazolinone hydrazone N-sulfonyl benzenesulfonate monosodium (MBTHSB) combined with 8-anilino-1 naphthalene sulfonic acid ammonium (ANS). In certain embodiments, the dye couple MBTHSB-ANS is preferred.

In yet other embodiments of colorimetric tester, signal producing systems that produce a fluorescent detectable product (or detectable non-fluorescent substance, e.g. in a fluorescent background) may be employed, such as those described in Kiyoshi Zaitsu, Yosuke Ohkura, New fluorogenic substrates for Horseradish Peroxidase: rapid and sensitive assay for hydrogen peroxide and the Peroxidase, Analytical Biochemistry (1980) 109, 109-113. Examples of such colorimetric reagent test strips suitable for use with the subject invention include those described in U.S. Pat. Nos. 5,563,042; 5,753,452; 5,789,255, herein incorporated by reference.

Electrochemical Sensor Variations

Instead of using a colorimetric sensor as described above, the present invention may also employ an electrochemical sensor. In the inventive tester the electrochemical sensor configuration presented in FIG. 2 may be exchanged for the membrane/matrix shown in FIG. 1A (and elsewhere) as indicated by the arrows. (In which case, aperture 40 should be omitted—unless a pair of electrode substrates as discussed below are provided.)

Typically, an electrochemical sensor comprises at least a pair of opposing electrodes. FIG. 2 shows a first electrode 54 that is preferably formed by a metallic coating 56 applied to frame 4—though a separate substrate applied to the backing may be employed. The second electrode 58 is shown in connection with an upper substrate or panel 60. In principle, the entire panel may be made of the metal.

However, like the frame, it preferably comprises plastic, that is coated with metal. Any convenient inert material may be used to form substrate(s), where typically the material is a rigid material that is capable of providing structural support to the electrode and to the electrochemical test strip as a whole. Suitable materials that may be employed as the backing substrate include plastics, e.g., polyester (PET), polyethylene terephthalate, glycol modified (PETG), polyimide, polycarbonate, polystyrene, silicon, ceramic, glass, and the like. Preferably, panel 60 comprises Mylar plastic film.

At least the surfaces of electrodes facing each other are comprised of a conductive layer such as a metal, where metals of interest include palladium, gold, platinum, silver, iridium, stainless steel and the like as well as carbon (conductive carbon ink) and doped tin oxide. One conductive layer is preferably formed by sputtering a thin layer of gold (Au), the other by sputtering a thin layer of palladium (Pd). Alternately, the electrodes may be formed by screen printing a selected conductive pattern, including conductive leads, with a carbon or metal ink on the backing surfaces. An additional insulating layer may be printed on top of this conductive layer which exposes a precisely defined pattern of electrodes. However formed, after deposition of conductive layers, the surface may be subsequently treated with a hydrophilic agent to facilitate transport of a fluid sample into the reaction zone therebetween.

In electrochemical biosensor embodiments of the present invention, the thickness of the any substrate material typically ranges from about 25 to 500 μm and usually from about 50 to 400 μm, while the thickness of the metal layer typically ranges from about 10 to 100 nm and usually from about 10 to 50 nm.

As mentioned above, the electrodes generally face each other and are separated by only a short distance, such that the spacing between the electrodes is extremely narrow. One or more spacers 62 internal to the substrate layer may be provided to define the requisite space. The thickness of spacer layer 62 may range from 10 to 750 μm and is often less than or equal to 500 μm, and usually ranges from about 25 to 175 μm. Any spacer layer preferably has double-sided adhesive to capture the adjacent electrodes. Spacer layer 12 may be fabricated from any convenient material, where representative suitable materials include polyethylene terephthalate, glycol modified (PETG), polyimide, polycarbonate, and the like.

In certain embodiments, spacer layer 62 is configured or cut-out so as to provide a reaction zone or area 64, where in many embodiments the volume of the reaction area or zone typically has a volume in the range from about 0.01 to 10 μL, usually from about 0.1 to 1.0 μL and more usually from about 0.05 to 1.0 μL. Spacer layer 62 may define any appropriately shaped reaction area, (e.g., circular, square, triangular, rectangular or irregular shaped reaction areas).

Regardless of reaction zone configuration, a reagent coating is provided. It preferably comprises a redox reagent system or composition selected to interact with targeted components in the fluid sample during an assay of the same.

In the case of the variation shown, reagent system 66 is deposited on the conductive layer 56, serving as electrode 54. This protion serves as a counter/reference electrode and top electrode 58 serves as the working electrode of the electrochemical cell. However, in other embodiments, depending on the voltage sequence applied to the cell, the role of the electrodes can be reversed. In case of a double pulse voltage waveform, each electrode acts as a counter/reference and working electrode once during the analyte concentration measurement.

Reagent systems of interest typically include an enzyme and a redox active component (mediator). The redox component of the reagent composition, when present, is made up of one or more redox agents. A variety of different redox agents (i.e., mediators) are known in the art and include: ferricyanide, phenazine ethosulphate, phenazine methosulfate, pheylenediamine, 1-methoxyphenazine methosulfate, 2,6-dimethyl-1,4-benzoquinone, 2,5-dichloro-1,4-benzoquinone, ferrocene derivatives, osmium bipyridyl complexes, ruthenium complexes, and the like. In many embodiments, the redox active component of particular interest is ferricyanide, and the like. The enzyme of choice may vary depending on the analyte concentration which is to be measured. For example, suitable enzymes for the assay of glucose in whole blood include glucose oxidase or dehydrogenase (NAD or PQQ based). Suitable enzymes for the assay of cholesterol in whole blood include cholesterol oxidase and esterase.

Other reagents that may be present in the reaction area include buffering agents (e.g., citraconate, citrate, malic, maleic, phosphate, “Good” buffers and the like); divalent cations (e.g., calcium chloride, and magnesium chloride); surfactants (e.g., Triton, Macol, Tetronic, Silwet, Zonyl, and Pluronic); and stabilizing agents (e.g., albumin, sucrose, trehalose, mannitol and lactose).

Examples of electrochemical biosensors suitable for use with the subject invention include those described in copending U.S. application Ser. Nos. 09/333,793; 09/497,304; 09/497,269; 09/736,788 and 09/746,116, the disclosures of which are herein incorporated by reference.

To make use of the electrochemical biosensor, sample may be introduced to the reaction zone through pores or ports 68 in substrate 60, preferably resembling a mesh or grid in this area. To produce an overall configuration that facilitates fluid passing through the holes into the sensor reaction zone spacers 62 may be omitted or be very thin and/or hydrophilic material or hydrophilic coating(s) may be employed. Alternately, or additionally, an absorptive pad, panel, mesh or membrane 88 may be employed to capture and/or direct sample acquisition. Furthermore, alternate electrode/sensor arrangements or features as taught in U.S. Pat. Nos. 5,508,171; 5,651,869 and/or 6,284,125 may be employed, the disclosures of which are herein incorporated by reference. In general, the amount of physiological sample (e.g., blood or interstitial fluid as obtained using blade(s) 6) that is introduced into the reaction area of the test strip may vary, but generally ranges from about 0.1 to 10 μl, usually from about 0.3 to 0.6 μl.

The component to be analyzed is allowed to react with the redox reagent coating to form an oxidizable (or reducible) substance in an amount corresponding to the concentration of the component to be analyzed (i.e., analyte). The quantity of the oxidizable (or reducible) substance present is then estimated by an electrochemical measurement.

The measurement that is made may vary depending on the particular nature of the assay and the device with which the electrochemical test strip is employed (e.g., depending on whether the assay is coulometric, amperometric or potentiometric). Measurement is preferably accomplished by way of a meter probe element inserted between the electrode members to contact their respective interior surfaces. Usually, measurement is taken over a given period of time following sample introduction into the reaction area. Methods for making electrochemical measurements are further described in U.S. Pat. Nos. 4,224,125; 4,545,382; and 5,266,179; as well as WO 97/18465 and WO 99/49307 publications.

Following detection of the electrochemical signal generated in the reaction zone, the amount of the analyte present in the sample is typically determined by relating the electrochemical signal generated from a series of previously obtained control or standard values. In many embodiments, the electrochemical signal measurement steps and analyte concentration derivation steps, are performed automatically by a device designed to work with the test strip to produce a value of analyte concentration in a sample applied to the test strip. A representative reading device for automatically practicing these steps, such that user need only apply sample to the reaction zone and then read the final analyte concentration result from the device, is further described in co-pending U.S. application Ser. No. 09/333,793 filed Jun. 15, 1999.

In certain variations, the spacer incorporated in the electrochemical cell is set back from the leading edge of each electrode. This provides a space for receipt of the meter probe. In addition, the cell may be setup so that substrate (or a substrate associated with frame 4 provide offset leading edges.

Though the invention has been described in reference to certain examples, optionally incorporating various features, the invention is not to be limited to the set-ups described. The invention is not limited to the uses noted or by way of the exemplary description provided herein. It is to be understood that the breadth of the present invention is to be limited only by the literal or equitable scope of the following claims. That being said, I claim:

1. A tester device for obtaining and testing a biological sample, said tester comprising:
a frame, a blade and a biosensor,
wherein said blade and said biosensor are discrete members carried by said frame at spaced-apart locations.

2. The device of claim 1, wherein only one blade is provided.

3. The device of claim 1, wherein at least one blade is provided.

4. The device of claim 1, wherein said biosensor is a colorimetric sensor.

5. The device of claim 1, wherein said biosensor is an electrochemical sensor.

6. The device of claim 1, wherein said biosensor is carried on a face of said frame.

7. The device of claim 6, wherein said blade is carried on said face of said frame.

8. The device of claim 7, wherein said blade is positioned on a wedge member carried by said face.

9. The device of claim 8, wherein said wedge comprises a land adjacent said blade, said land having a width of about 0.5 mm to 2.0 mm.

10. The device of claim 6, wherein said blade is positioned between said face and a front wall.

11. The device of claim 10, wherein a region between said face and said front wall is relieved.

12. The device of claim 1, wherein said frame comprises a pair of opposing side walls.

13. The device of claim 12, wherein said frame further comprises a front wall.

14. The device of claim 1, wherein said blade comprises a triangular member.

15. The device of claim 14, wherein said blade further comprises at least one side portion and at least one champhered portion.

16. The device of claim 15, wherein said at least one side portion is a substantially planar portion substantially perpendicular said face of said frame.

17. The device of claim 1, wherein said blade is between about 0.1 and 1.0 in height.

18. The device of claim 16, wherein said blade has a profile defining an angle between 60 and 135°.

19. The device of claim 1, where said frame and said blade are injection molded together.

20. The device of claim 19, wherein a wedge for said blade is injection molded together with said frame and blade.

21. The device of claim 1, wherein said frame includes a body portion for mounting said biosensor in a convex or outwardly-facing orientation.

22. The device of claim 21, wherein said frame has a curved body.

23. A method of tester use comprising:
providing a tester as described in any of claims 1-22;
passing said tester over a skin surface of a subject, wherein in said passing of said tester, said blade cuts said skin surface to produce a laceration and said biosensor is placed in contact with said laceration,
collecting a biological sample with said biosensor from said laceration, and
testing said sample for analyte concentration.

24. The method of claim 23, further comprising testing said biological fluid collected.

25. The method of claim 23, wherein said biological fluid is blood.

26. The method of claim 23, wherein said biological fluid is interstitial fluid.

27. The method of claim 23, wherein said passing comprises angling a front end of said tester to contact said digit and then returning said tester from said angled orientation.

28. The method of claim 3, further comprising providing a stack of a plurality of testers as described in any of claims 1-22.

29. The method of clam 27, further comprising, performing the acts of claim 22 for each tester provided in said stack.

30. The method of claim 23, wherein said analyte is glucose.
